(19) United States
(12) Patent Application Publication
(10) Pub. No.: US 2001/0051672 A1
(43) Pub. Date: Dec. 13, 2001

(54) Dental material based on cationically polymerizable monomers

(76) Inventors: Philipp Albert, Hanau (DE); Corinna Gall, Schluchtern (DE); Gerd Lohden, Hanau (DE); Harald Rentsch, Hanau (DE)

Correspondence Address: SMITH, GAMBRELL & RUSSELL, LLP, Suite 800, 1850 M Street, N.W., WASHINGTON, DC, 20036, US

(21) Appl. No.: 09/821,757

(22) Filed: Mar. 30, 2001

(30) Foreign Application Priority Data
Mar. 31, 2000 (DE) 100 16 323.8

(51) Int. Cl. C08K 3/34; C08K 3/40; A61K 6/08

(52) U.S. Cl. 523/116; 524/492; 524/494; 524/450

(57) ABSTRACT

Dental materials based on cationically polymerizable monomers as binders, a polymerization initiator, and based on the dental material, 1-95 wt % of at least one inorganic filler, wherein the binder contains monomers of formula (I):

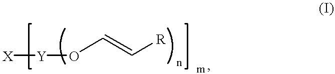

wherein R represents hydrogen or a methyl or ethyl group; X and Y independently represent an unsubstituted or substituted aliphatic, cycloaliphatic, or aromatic residue with 1-100 carbon atoms, wherein one or more CH₂ groups can be replaced by O, C═O, —CO₂, —SiR¹₂—, and/or —SiR¹₂O—, wherein R¹ independently denotes an alkyl or alkoxy or aryl residue with 1-10 C atoms; n represents a whole number of 1 to 3; and m represents a whole number of 2-5. The new dental compositions have especially low loss of volume, caused by curing, and particularly good characteristics, and short polymerization times.

### INTRODUCTION AND BACKGROUND

The present invention relates to dental materials comprising cationically polymerizable monomers and a method for their production.

Polymerizable dental materials of the current state of the art contain mostly bi- or polyfunctional acrylates and methacrylates, which are radically cured.

Such materials are, for example, described in EP-A-0 091 990. A great disadvantage of the known polymerizable dental compositions is polymerization shrinkage, which can cause secondary caries due to the formation of boundary cracks, for example, when used as filling material. To improve this shrinkage, monomers proposed in EP-A-0 754 675 are used, which have liquid-crystalline characteristics. This measure leads to a perceptible improvement in reducing the loss of volume, but it is not sufficient for many purposes.

In addition, polymerizable compositions based on epoxides are known, which can be used as dental material. Such dental materials are described, for example, in WO 96/13 538, WO 95/30 402, and WO 98/22 521.

The disadvantage of these epoxides is, first of all, the low rate of curing. This leads, among other things, to a longer process for introduction of the filling material into the cavity exposed by the dentist. Another disadvantage of dental materials based on epoxides is their low storage stability. Epoxides can be polymerized both cationically with acids and also anionically by bases. Therefore, it is not possible to substantially increase the storage lifetime by the addition of inhibitors. Whereas methacrylates can be stored by the addition of radical traps, both the addition of bases and also of acids leads to a polymerization of the epoxides.

Another disadvantage is the relatively high viscosity of epoxides, which can be attributed to their polarity. As a result of this high viscosity, only relatively low fractions of fillers can be incorporated or one must use monomers for dilution. This leads inevitably to relatively unfavorable mechanical characteristics of the cured dental materials.

Moreover, many epoxides are cytotoxic, to a high degree. In-vitro tests have shown that most of these monomers have mutagenic characteristics.

In view of the state of the art as acknowledged and discussed herein, it was, therefore, an object of the invention to provide an improved dental material, which, in particular, exhibits a reduced shrinkage and a high rate of curing.

Furthermore, it is another object of the invention to provide binders which exhibit high storage stability and a low toxicity. It is yet a further object of the invention to be able to easily obtain and/or simply produce the components of the binder, in particular, the monomers.

Moreover, all requirements of modem dental material with regard to transparency, polishing capacity, compressive strength, water absorption, wear resistance, flexural strength, X-ray opacity, and so forth, should be met to a particularly high degree.

### SUMMARY OF THE INVENTION

The above and other objects of the present invention can be attained with a dental material formed from at least one cationally polymerizable monomer as a binder, a polymerization initiator and, based on the dental material, 1 to 95% by weight of at least one inorganic filler.

The binder of the present invention contains at least one monomer of formula (I):

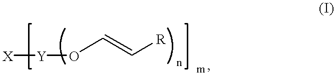

wherein R represents hydrogen, methyl or ethyl group; X and Y independently represent an unsubstituted or substituted aliphatic, cycloaliphatic, or aromatic residue with 1-100 carbon atoms, wherein one or more CH₂ groups can be replaced by O, C═O, —CO₂—, —SiR¹₂—, and/or —SiR¹₂O—, wherein R¹ independently denotes an alkyl or alkoxy or aryl residue with 1-10 C atoms; n represents a whole number from 1-3; and m represents a whole number from 2-5.

It is possible according to the invention to create a dental material based on the above cationically polymerizable monomers as binders, a polymerization initiator to be described herein, and based on the dental material, 1-95 wt % of at least one inorganic filler as set forth below, which material exhibits less shrinkage with a simultaneously high storage capacity than do prior known compositions.

The following advantages, among others, are obtainable in accordance with the present invention:

The cured dental compositions exhibit a high transparency, an excellent polishing capacity, a very good compressive strength, an extremely low water absorption, a high wear resistance, a very high flexural strength, and a high X-ray opacity.

The dental material exhibits a high storage lifetime, a high rate of curing, a good processability, and low toxicity.

The polymerization needs merely to be initialed for it to continue independently thereafter.

The shrinkage of the dental materials, in accordance with the invention, is very low.

Monomers of formula (I) can be obtained in a relatively simple manner and at low cost.

Within the framework of the present invention, the term “dental material” designates materials for tooth fillings, inlays or onlays, dental cements, glass ionomer cements, composite polymers, composites, facing materials for crowns and bridges, materials for false teeth, dentin bondings, base filling materials, root filling materials, or other materials for prosthetic, preserving, and preventive dentistry. In particular, composites for applications in dentistry and dental technology, sealing materials, self-curing composites, stump synthesis materials, facing plastics, highly and normally filled dual cements, and normally filled, fluoride-containing dental lacquers also fall under the term “dental material.”

Usually, these dental materials comprise binders, polymerization initiators, and inorganic fillers.

The binder of dental materials, in accordance with the invention, contains monomers of formula (I):

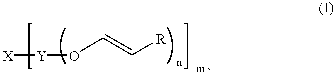

wherein R represents hydrogen, a methyl or ethyl group; X and Y independently represent an unsubstituted or substituted aliphatic, cycloaliphatic, or aromatic residue with 1-100 carbon atoms, wherein one or more CH₂ groups can be replaced by O, C═O, —CO₂—, —SiR¹₂—, and/or —SiR¹₂O—, wherein R¹, independently, denotes an alkyl or alkoxy or aryl residue with 1 to 10 C atoms; n represents a whole number from 1-3, preferably, 1 or 2; and m represents a whole number from 2-5, preferably 3 or 4.

A preferred group of monomers of formula (I) is voluminous but at the same time, rigid. Voluminous monomers generally exhibit a particularly low polymerization shrinkage. High-molecular substances, however, increase the viscosity of the binder, so that in this way, the filler content of the dental material is limited.

Accordingly, monomers of formula (I) have aromatic groups, which, for example, are derived from benzene, biphenyl, diphenyl ether, bisphenol A (2,2-bis(4-hydroxyphenyl)propane), and/or diphenyl carbonate. These groups can also be derived from condensed, aromatic ring systems, such as naphthalene, acenaphthylene, acenaphthene, fluorene, phenanthrene, anthracene, fluoranthene, pyrene, benz[a]anthracene, chrysene, and/or have hetero atoms, in particular, sulfur and/or oxygen. Examples of this are benzofurans, benzothiophenes, dibenzofurans, and dibenzothiophenes.

These groups can be present, in particular, in the form of aromatic polyester, aromatic polyether groups, and/or polycarbonate groups, wherein the number of carbon atoms should not exceed the aforementioned values.

Moreover, cyclic, in particular, polycyclic alkyl groups, which can be derived, among others, from norbornane, adamantane, norcarane, pinane, tricyclene, are also particularly suitable.

Another group of monomers, which can be present in the binder, are flexible monomers. Among others, siloxane, alkyl, or polyether residues can belong to groups which have these monomers.

In a very particularly preferred embodiment, both groups; i.e. rigid and flexible, are used as a mixture. By this measure, the mechanical characteristics can be improved in a nonforeseeable manner, wherein, in particular, tough dental materials are obtained. Depending on the application, the mixing ratio can fluctuate within wide ranges. In general, however, mixtures, are used, in which the ratio of flexible monomer to rigid monomer is in the range of 10:1-1:100, in particular, 1:1-1:10.

A similar effect can be obtained in that monomers have both flexible as well as rigid groups in the molecule. Thus, for example, the residue X can be rigid, where Y is flexible.

Monomers of formula (I) are at least bifunctional, wherein the functionality results from the product of n and m. Preferably, this product is ≧3, with particular preference, in the range of 4-6. The higher the functionality of the monomer, the higher will be the probability of incorporation into the matrix.

Furthermore, a high functionality is necessary, so as to attain a high crosslinking density of the plastic matrix. This leads to materials with a high strength and durability. With increasing crosslinking density, the plastics tend, however, to become brittle, which, under certain circumstances can be fraught with disadvantages.

Examples of preferred groups, which have the residue X of a compound of formula I, are, among others:

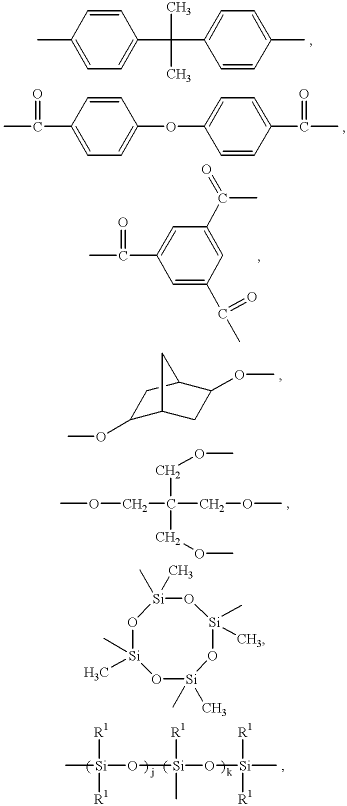

wherein R¹ independently represents an alkyl or alkoxy residue with 1-6 C atoms; j and k independently represent whole numbers in the range from 1 to 10. Special examples and preferred embodiments of the residues Y are the following:

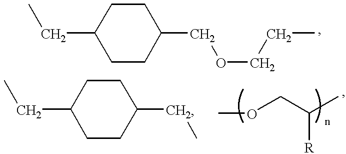

wherein R represents hydrogen or methyl; and n represents a whole number in the range from 1-10.

The residues X and Y can also have substituents. Among others, alkyl groups such as methyl, ethyl, propyl, butyl, 2-propenyl, and 2-butenyl; aryl residues, such as phenyl, cumyl, xylyl, and naphthyl; alkoxy residues, such as methoxy, ethoxy, propoxy, belong to these substituents.

These residues can, moreover, comprise halogen-containing groups, ester groups, hydroxy groups, ether groups, amino groups, amide groups, urethane groups, and carbonic acid groups. However, the fraction of basic groups—among others, amino groups, amide groups, and urethane groups—is limited in that these groups can have a disadvantageous effect on the cationic polymerization.

Special examples of monomers of formula (I) are, among others, 1,3,5-benzenetricarboxylic acid tris-4-(ethenyloxy)butanol ester; butane diacid-bis(4-ethenyloxy)butanol ester; bis(4-(ethenyloxy)methyl)cyclohexyl)methyl)pentanedioate; 2,2-bis(4,1-phenyloxy-4-((ethenyloxy)methyl)cyclohexyl)methyl)propane; diphenyl ether-4,4′-dicarboxylic acid (4-((ethenyloxy)methylcyclohexyl)methanol diester; diphenyl ether-4,4′-dicarboxylic acid-2-(ethenyloxy)ethanol diester, and 1,3,5,7-tetra((4-((1-propenyloxy)methyl)cyclohexyl)methoxyethyl)-1,3,5,7-tetramethylcyclotetrasiloxane.

Dental materials should have, in a preferred embodiment, an index of refraction which is approximately that of the natural material. It is also advantageous if the index of refraction of the matrix approximately corresponds to that of the filler, since otherwise the turbidity of the material increases. This could also lead to problems with a possible photocuring of the dental material. In order to increase the index of refraction, the fraction of aromatic groups in the binder can be increased. Moreover, sulfur atoms also increase this value, wherein these atoms can be present, for example, because of thioether, thioester, and/or thio groups in monomers of the binder.

Particularly preferred monomers of formula (I) have a molecular weight in the range of 300-3000. It was particularly surprisingly determined that monomers that fulfill this criterion have a particularly low shrinkage, wherein the cured dental material exhibits excellent mechanical characteristics at the same time, such as high pressure resistance, high modulus of elasticity, and high flexural strength, without, however, being brittle.

Some monomers of formula (I) are polymers or oligomers. As a result of their production, these compounds generally have a molecular weight distribution. The previously indicated values of the molecular weight refer, in these cases, to the weight average. This can be determined, for example, by viscosimetry.

The aforementioned monomers can, for the most part, be commercially obtained; moreover, they can be obtained synthetically in a manner which is, in fact, known. Here, the aforementioned residues X and Y can serve as reference points. Persons skilled in the art can, moreover, find helpful instructions, for example, in Ullmann's Encyclopedia of Industrial Chemistry, 5th Edition.

The production of vinyl compounds from vinyl halides and alkoxides was described, in particular, from W. Reppe et al., Justus Liebigs Ann. Chem. 601 (1956), 81-110. Moreover, transvinylation reactions are known, in which a vinyl group of a vinyl ether or ester is transferred to an alcohol or an acid. The following literature references are mentioned, by way of example for many: N. D. Field et al.: “Vinyl Ethers,” in High Polymers, Vol. 24, Wiley, New York, 1971, pp. 365-411; S. A. Miller: “Vinyl Ethers,” in Acetylene, Its Properties, Manufacture and Uses, Vol. 2, Ernest Benn Ltd., London, 1966, pp. 198-231; D. H. Lorenz: Encyclopedia of Polymer Science and Technology, Vol. 14, Interscience, New York, 1971, pp. 504-510.

Moreover, vinyl groups can be obtained from allyl groups by isomerization reactions. The isomerization can take place both by the addition of a base and also by the action of Ru catalysts, such as (Ph₃P)₃RuCl₂.

The aforementioned siloxanes can, for example, be obtained from the corresponding hydrogen siloxanes by hydrosilylation with (1-propenoxy)vinyloxyalkanes. In that case, the vinyloxy group reacts mainly with the Si—H bond of the hydrogen siloxane. For the catalysis of this reaction, Pt, Rh, and/or Pd compounds are generally used. Among others, Karsted and Wilkinson catalysts are known. (1-Propenoxy)vinyloxyalkanes can, for example, be obtained by isomerization from allyloxyvinyloxyalkanes.

Preferably, the viscosity of binder is 1 mPa·s to 6 Pa·s, preferably 1 mPa·s to 1 Pa·s without this implying a limitation. The lower limiting value is produced from the volatility of the binder. If the viscosity is greater than 6 Pa·s, then only a very limited quantity of inorganic filler can be incorporated. A high filler content, however, leads to low shrinkage, excellent mechanical characteristics, and to a very good polishing capacity of the cured material.

To influence the viscosity, the binder can, moreover, comprise monofunctional monomers, which can be copolymerized cationically with compounds of formula (I).

Among others, vinyl esters, such as vinyl acetate, vinyl ethers, such as propylvinyl ether, butylvinyl ether, 2-methylpropylvinyl ether, pentylvinyl ether, hexylvinyl ether, ((1-propenoxy)ethyl)trimethylsilane, ((1-propenoxy)ethyl)triethylsilane, and vinyl aromatics, such as styrene, substituted styrenes with an alkyl substituent in the side chain, such as α-methylstyrene and α-ethylstyrene, substituted styrenes with an alkyl substituent on the ring, such as vinyltoluene, and p-methylstyrene, halogenated styrenes, such as monochlorostyrenes, dichlorostyrenes, tribromostyrenes, and tetrabromostyrenes.

A polymerization initiator is added for the curing of the dental material. Preferably, the polymerization takes place cationically. Accordingly, particularly preferred initiators contain Lewis or Bronsted acids or compounds, which release such acids, such as BP₃ or its ethereal adducts (BF₃.THF, BF₃.Et₂O, and so forth), AlCl₃, FeCl₃, HPF₆, HAsF₆, HSbF₆, HBF₄to which under certain circumstances, a halogenated carbon, such as triphenylchloromethane, is added or substances which after irradiation with UV or visible light or with heat and/or pressure trigger the polymerization, such as (eta-6-cumene)(eta-5-cyclopentadienyl)iron hexafluorophosphate, (eta-6-cumene)(eta-5-cyclopentadienyl)iron tetrafluoroborate, substituted diaryl iodonium salts and triaryl sulfonium salts.

Usually, accelerators or sensitizers, such as peroxy compounds, in particular, of the perester type, benzoin derivatives, benzil compounds, or acylphosphine oxides, are added to these initiators. The ratio of initiator to accelerator can be varied within broad limits, from 1:0.001-1:10; preferably, however, a ratio of 1:0.1-3:6 is preferably used.

Particularly preferred polymerization initiators contain iodonium salts as initiators and benzoin derivatives, such as benzoin, α-methylbenzoin methyl ether, α-dicarbonyl compounds, such as 2,3-butanedione, camphor quinone, benzil and its derivatives, such as ω,ω-dimethoxy-ω-phenylacetophenone, α-hydroxyalkylphenone derivatives, such as 1-benzolycyclohexan-1-ol, and acylphosphine oxide compounds, such as benzoyldiphenylphosphine oxide, trimethylbenzoyldiphenylphosphine oxide (others are described in EP 0 073 413) as sensitizers. To some extent, amines are added as additional coinitiators.

Particularly suitable diaryl iodonium compounds are, for example, diphenyl iodonium tetrafluoroborate, diphenyl iodonium hexafluorophosphate, bis(4-methylphenyl) iodonium hexafluorophosphate, dinaphthyl iodonium hexafluoroantimonate, and phenyl-4-methylphenyl iodonium hexafluoroantimonate.

The polymerization initiators containing iodonium salts, which generally contain sensitizers, can be frequently initiated with visible light in the wavelength range of 380-500 nm. Surprisingly, it was determined that these initiators cure compounds of formula (I) in a particularly effective and rapid manner. In this case, it is not necessary to irradiate the material during the entire curing time, since after a sufficient initiation, the dental material is completely cured. This characteristic is particularly useful during the introduction of fillings into the prepared cavity.

The use of inorganic fillers in dental materials is, in fact, known. The fillers are used, in particular, to improve mechanical characteristics. Among others, quartzes, ground glass, aerosils (silica), spherical SiO₂ particles, which are optionally coated with titanium dioxide, zeolites, ormocers, ormosils, hard-to-dissolve fluorides, such as CaF₂, YF₃, silica gels and pyrogenic silica or their granules are used, to a large extent, as fillers. Moreover, the dental materials, in accordance with the invention, can also have organic fillers, in particular, fibers. These fillers can generally be commercially obtained.

In particular, heavy atoms, such as Y and Zr, increase X-ray opacity. Therefore, fillers which have these atoms are preferred.

For a better incorporation into the polymer matrix, these fillers can be treated with adhesion-improving agents. Among others, silanes, such as ((1-propenoxy)ethyl)trimethoxysilane, ((1-propenoxy)ethyl)triethoxysilane, or (1-propenoxy)ethyltrichlorosilane, are suitable for this purpose.

Without creating a limitation hereby, these fillers should generally exhibit a particle size in the range of 0.02-100 μm preferably 0.05-10 μm, and with very particular preference, 0.1-5 μm. The form of the fillers is not subject to any particular limitation. Accordingly, they can be, for example, spherical, splinter-shaped, plate-shaped, and/or in the shape of fibers.

These fillers can be used singly or as mixtures, wherein the use of mixtures makes possible, under certain circumstances, improvements with regard to aesthetics and a further improvement of mechanical characteristics.

The filler content of the dental materials, including the ormocers, in accordance with the invention, lies in the range of 1-95 wt %, preferably 50-90 wt %, and with very particular preference of 65-90 wt %, based on the total weight. A high filler content leads to slight shrinkage, excellent mechanical characteristics, and to a good polishing capacity of the cured material. On the other hand, sufficient binder for curing must be present in the dental material. The more uniformly the binder can be incorporated in the filler, the higher the filler content used.

Moreover, the dental materials of the invention under consideration can have auxiliaries. Among others, stabilizers, pigments, or diluents can be used as auxiliary ingredients.

A particularly preferred dental material of the invention under consideration consists, for example, of the following:

| | |
|---|---|
| 4.98-95 wt % | binder; |
| 0.02-10 wt % | polymerization initiator; |
| 1-95 wt % | filler; and |
| 0-20 wt % | usual additives, based on the total |
| | weight of the dental material. |

The dental material, in accordance with the invention, can be produced in a manner, which is, in fact, known. The components are hereby mixed, wherein the filler is preferably added in portions.

The cured dental material exhibits excellent characteristics with respect to flexural strength, in accordance with DIN 53 452, the modulus of elasticity, in accordance with DIN 53 457, compressive strength, durability, and wear resistance; thus, the flexural strength of preferred embodiments is generally ≧100 N/mm²; and the modulus of elasticity, preferably ≧8000 N/mm².

Moreover, the dental materials of the invention under consideration have an excellent polishing capacity, low water absorption, and excellent aesthetic characteristics, in particular, with regard to transparency and the index of refraction.

### DETAILED DESCRIPTION OF INVENTION

The examples and comparative examples below will serve as a more precise explanation of the invention, without being limited to them. The following percentages refer to the total weight, unless otherwise noted.

### EXAMPLE 1

Production of 1,3,5,7-tetra-((4((1-propenyloxy)methylcyclohexyl)methoxyethyl)-1,3,5,7-tetramethylcyclotetrasiloxane (DEB 461)

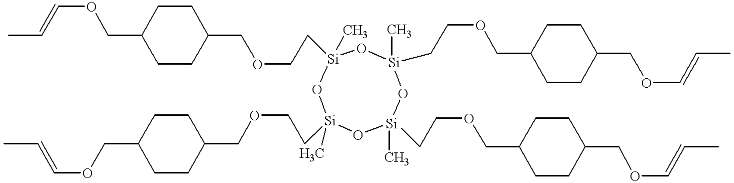

Into a 500 ml three neck flask there is placed 0.5 mol (84.5 g) 4-(hydroxymethyl)-1-(vinyloxymethyl)cyclohexane, 0.65 mol (78.64 g) allylbromide, 0.65 mol (26 g) NaOH, 0.31 mol (10 g) tetra-n-butylammonium bromide, and 200 mL toluene. The flask is equipped with a thermometer, stirrer, and reflux condenser. After 15 min of stirring, under a nitrogen atmosphere, the mixture is heated to 90° C. for 16 h. Afterwards, cooling to room temperature is carried out, and the mixture is placed in 300 mL water. The organic phase is separated and washed twice with water. Subsequently, the solvent is removed by distillation under reduced pressure. The residue is purified by means of chromatography, wherein silica gel and a 1:9 mixture of ethyl acetate and hexane is used.

The allylvinyl alkane thus obtained is subsequently converted into the 1-propenyl derivative by isomerization. Here, the previously obtained product is placed in a round-bottom flask, which is equipped with a reflux condenser and stirrer. After the addition of 0.64 μmol (0.06 g) (Ph₃P)₃RuCl₂, the mixture is heated to 130° C. for 8 h. The product obtained is subsequently distilled under reduced pressure (93° C. under approximately 26 Pa). All total, 0.435 mol (91.8 g) 4-(1-propenyloxymethyl)-1-(vinyloxymethyl)cyclohexane were obtained (approximately 87% yield).

The 4-(1-propenyloxymethyl)-1-(vinyloxymethyl)cyclohexane is subsequently reacted with S-H-functional 1,3,5,7-tetramethylcyclotetrasiloxane.

0.435 mol (91.8 g) of the divinyl ether, 0.1 mol (24 g) of the cyclosiloxane, and 100 mL dry toluene are added to the mixture in a round-bottom flask. Subsequently, 5 μL Karsted catalyst are added (3% Pt complex in xylene, in accordance with U.S. Pat. No. 3,814,730). The mixture is stirred first at 50° C. under a nitrogen atmosphere. Subsequently, the mixture is heated to 100° C. by released heat of reaction. After 16 h, the reaction is ended. Afterwards, the educts and the solvent are separated by means of distillation, wherein residues are removed in a high vacuum.

The product obtained (DEB 461) was characterized by means of NMR spectroscopy and elementary analysis and used without further purification. The yield is approximately 98% (105.8 g).

### EXAMPLE 2

11.88 g DEB 461, as produced in Example 1, are mixed with 0.0839 g (0.7%) iodonium salt (4-(n-octyloxy)phenyl)phenyl iodoniumhexafluoroantimonate)), 0.0248 g sensitizers (acylphosphine oxide, Igacure®819), and 0.0024 g (0.002%) stabilizer (BHT Ralox®).

The mixture thus produced is irradiated with blue light for 20 sec, wherein this is completely cured. The plastic matrix cured in this way is investigated for its mechanical characteristics and the volume decrease, which occurs because of the polymerization (shrinkage). The results obtained are shown in Table 1.

### EXAMPLE 3

In order to produce a dental material from the mixture obtained in Example 2, a total of 0.645 g aerosil is first added, in two portions to 11.99 g of the curable monomer mixture. Subsequently, glass, silanized with vinyloxyethyl-N-(3-(triethoxysilyl)propyl)carbamate (VES), is kneaded in. The addition of a total 24.81 g takes place in new steps (1: 9.99 g; 2: 5.04 g; 3: 2.49 g; 4: 1.99 g; 5: 2.07 g; 6: 1.63 g; 7: 0.83 g; 8: 0.54 g; and 9: 0.23 g), wherein a miniature kneader is used, with a kneading time of 10 min and a pressure of 180-210 mbar.

### Comparative Example 1

Production of 1,3,5,7-tetra-((4-((oxymethacroyl)methyl)cyclohexyl)methoxyethyl)-1,3,5,7-tetramethylcyclotetrasiloxane (DEB 502)

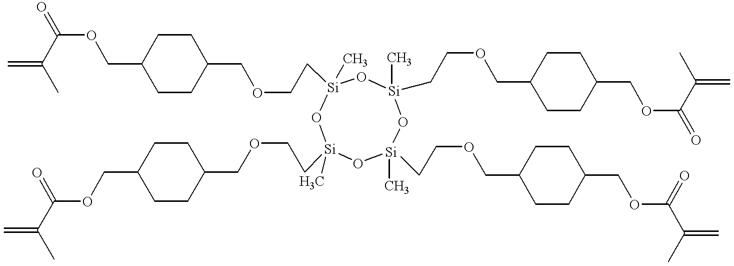

The production of DEB 502 is performed in a manner similar to the preparation of DEB 461. However, 4-(hydroxymethyl)-1-(vinyloxymethyl)cyclohexane is first reacted with 1,3,5,7-tetramethylcyclotetrasiloxane. Subsequently, the product is reacted with methacrylic acid anhydride to DEB 502.

### Comparative Example 2

Comparative Example 2 is carried out in a manner similar to Example 2, except that DEB 502 is used instead of DEB 461. Accordingly, a radical initiator system, consisting of camphor quinone and Speedcure EDB, is used.

The binder is cured, moreover, for 60 sec, in order to ensure a complete reaction. This plastic matrix is also investigated for their mechanical characteristics and shrinkage. The values obtained are given in Table 1.

### EXAMPLE 4

Example 4 is performed in the same manner as in Example 2, except that 11.88 g diphenyl ether 4,4′-dicarboxylic acid(4-vinyloxymethyl)cyclohexyl)methanol diester (CHMVEE) instead of DEB 461 were used, which was obtained on the basis of Example 1:

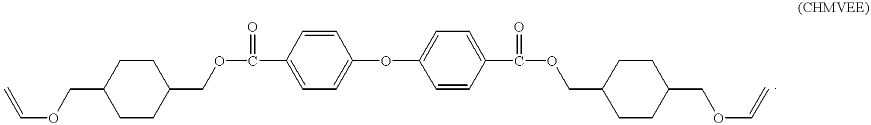

After curing, the plastic matrix is investigated for their mechanical characteristics and shrinkage. The results obtained are shown in Table 1.

TABLE 1
Mechanical characteristics of the plastic matrix.

| | Shrinkage | Flexural strength | E-Modulus |
|---|---|---|---|
| | (Δ V) | (DIN 53452) | (DIN 53457) |
| | [%] | [N/mm²] | [N/mm²] |
| Example 2 | 2.7761 | 50 | 1200 |
| Comparative example 2 | 5.2198 | 40 | 800 |
| Example 4 | 3.8315 | 80 | 2000 |

Further variations and modifications of the foregoing will be apparent to those skilled in the art and are intended to be encompassed by the claims appended hereto.

German priority application 100 16 323.8 is relied on and incorporated herein by reference.

We claim:

1. A dental material comprising at least one cationically polymerizable monomer as a binder, a polymerization initiator, and based on the dental material, 1-95 wt % of at least one inorganic filler, said binder containing at least one monomer of formula (I): 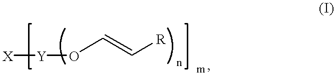
wherein R represents hydrogen, a methyl or ethyl group; X and Y independently represent an unsubstituted or substituted aliphatic, cycloaliphatic, or aromatic residue with 1-100 carbon atoms, wherein one or more CH₂ groups can be replaced by O, C═O, —CO₂, —SiR¹₂—, and/or —SiR¹₂O—, wherein R¹ independently represents an alkyl or alkoxy or aryl residue with 1-10 C atoms; n represents a whole number of 1-3; and m represents a whole number of 2-5.

2. The dental material according to claim 1, wherein the residue X is one or more of the following groups: 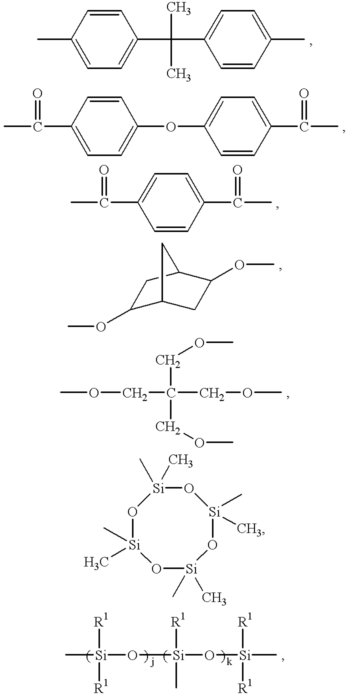
wherein R¹ independently represents an alkyl or alkoxy residue with 1-6 C atoms; j and k independently represent whole numbers in the range of 1-10.

3. The dental material according to claim 1, wherein the residue Y has one or more groups: 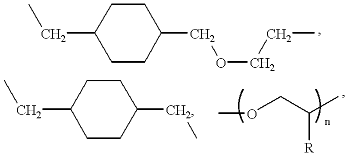
wherein R represents hydrogen or methyl; and n represents a whole number in the range of 1-10.

4. The dental material according to claim 2, wherein the residue Y has one or more groups: 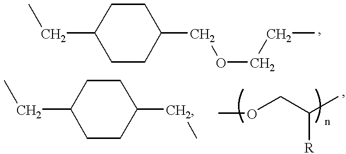
wherein R represents hydrogen or methyl; and n represents a whole number in the range of 1-10.

5. The dental material according to claim 1, wherein the monomers of formula (I) have a molecular weight in the range of 300-3000.

6. The dental material according to claim 1, wherein the binder has a viscosity in the range of 1 mPa·s to 1000 mPa·s.

7. The dental material according to claim 1, wherein the binder also has monofunctional vinyl ethers.

8. The dental material according to claim 1, wherein the polymerization initiator contains an iodonium salt and a sensitizer.

9. The dental material according to claim 1, wherein the polymerization initiator can be initiated by irradiation with visible light.

10. The dental material according to claim 1, wherein the filler is a member selected quartz, ground glass, silica gel, from the group consisting of silica, a zeolite, an ormocer, and mixtures of these substances.

11. The dental material according to claim 10, wherein the filler is treated with an adhesive.

12. The dental material according to claim 1, wherein the filler content is in the range of 50-90 wt % based on the total weight.

13. The dental material according to claim 1, wherein the filler content is in the range of 65-90 wt % based on the total weight.

14. The dental material according to claim 1 wherein the flexural strength thereof is ≧30 N/mm² in accordance with DIN 53 452, and/or the modulus of elasticity thereof is ≧500 N/mm² in accordance with DIN 53 457.

15. The dental material according claim 1, consisting of the following:

| | |
|---|---|
| 4.98-95 wt % | Binder; |
| 0.02-10 wt % | Polymerization initiator; |
| 1-95 wt % | Filler; and |
| 0-20 wt % | Usual additives, based on the total |
| | weight of the dental material. |

16. A method for the production of a dental material according to claim 1, comprising mixing components together.
